(19) United States
(12) Patent Application Publication
(10) Pub. No.: US 2022/0366151 A1
(43) Pub. Date: Nov. 17, 2022

(54) DOCUMENT CREATION SUPPORT APPARATUS, METHOD, AND PROGRAM

(71) Applicant: FUJIFILM Corporation, Tokyo (JP)

(72) Inventor: Keigo NAKAMURA, Tokyo (JP)

(73) Assignee: FUJIFILM Corporation, Tokyo (JP)

(21) Appl. No.: 17/867,674

(22) Filed: Jul. 18, 2022

Related U.S. Application Data
(63) Continuation of application No. PCT/JP2021/004366, filed on Feb. 5, 2021.

(30) Foreign Application Priority Data
Feb. 7, 2020 (JP) 2020-019954

(51) Int. Cl. G06F 40/56; A61B 6/00

(52) U.S. Cl. CPC G06F 40/56; A61B 6/463; A61B 6/5217

(57) ABSTRACT

A document creation support apparatus includes at least one processor, in which the processor is configured to derive properties for each of a plurality of predetermined property items in a structure of interest included in an image, generate a plurality of sentences describing the properties specified for at least one of the plurality of property items, and display each of the plurality of sentences, and display a described item, which is a property item of a property that is described in at least one of the plurality of sentences among the plurality of property items, on a display screen in an identifiable manner.

### CROSS REFERENCE TO RELATED APPLICATIONS

The present application is a Continuation of PCT International Application No. PCT/JP2021/004366, filed on Feb. 5, 2021, which claims priority to Japanese Patent Application No. 2020-019954, filed on Feb. 7, 2020. Each application above is hereby expressly incorporated by reference, in its entirety, into the present application.

### BACKGROUND

### Technical Field

The present disclosure relates to a document creation support apparatus, a method, and a program that support creation of documents in which medical sentences and the like are described.

### Related Art

In recent years, advances in medical devices, such as computed tomography (CT) apparatuses and magnetic resonance imaging (MRI) apparatuses, have enabled image diagnosis using high-resolution medical images with higher quality. In particular, since a region of a lesion can be accurately specified by image diagnosis using CT images, MM images, and the like, appropriate treatment is being performed based on the specified result.

In addition, image diagnosis is also made by analyzing a medical image via computer-aided diagnosis (CAD) using a learning model in which machine learning is performed by deep learning or the like, discriminating properties such as the shape, density, position, and size of structures of interest such as abnormal shadow candidates included in the medical images, and acquiring them as an analysis result. The analysis result acquired by CAD is associated with examination information such as a patient name, gender, age, and a modality that has acquired the medical image, and is saved in a database. The medical image and the analysis result are transmitted to a terminal of a radiologist who interprets the medical images. The radiologist interprets the medical image by referring to the transmitted medical image and analysis result and creates an interpretation report, in his or her own terminal.

Meanwhile, with the improvement of the performance of the CT apparatus and the MRI apparatus described above, the number of medical images to be interpreted is also increasing. However, since the number of radiologists has not kept up with the number of medical images, it is desired to reduce the burden of the image interpretation work of the radiologists. Therefore, various methods have been proposed to support the creation of medical sentences such as interpretation reports. For example, JP2019-153250A proposes various methods for generating a sentence to be included in an interpretation report based on keywords input by a radiologist and on information indicating a property of a structure of interest (hereinafter referred to as property information) included in an analysis result of a medical image. In the methods described in JP2019-153250A, a sentence relating to medical care (hereinafter referred to as a medical sentence) is created by using a learning model in which machine learning is performed, such as a recurrent neural network trained to generate a sentence from characters representing the input property information. By automatically generating the medical sentence as in the method described in JP2019-153250A, it is possible to reduce a burden on a radiologist at the time of creating a medical sentence such as an interpretation report.

It is preferable that the medical sentence such as the interpretation report appropriately expresses the property of a structure of interest included in the image, or reflects the preference of a reader such as an attending physician who reads the medical sentence. Therefore, there is a demand for a system in which, for one medical image, a plurality of medical sentences with different expressions are generated or a plurality of medical sentences describing different types of properties are generated and presented to a radiologist so that the radiologist can select the most suitable medical sentence. Further, in this case, it is desired to be able to ascertain which property information is described in each of the plurality of sentences.

### SUMMARY OF THE INVENTION

The present disclosure has been made in view of the above circumstances, and an object thereof is to make it easy to recognize whether or not there is a description of property information about a structure of interest included in an image in a sentence related to the image.

According to an aspect of the present disclosure, there is provided a document creation support apparatus comprising at least one processor, in which the processor is configured to derive properties for each of a plurality of predetermined property items in a structure of interest included in an image, generate a plurality of sentences describing the properties specified for at least one of the plurality of property items, and display each of the plurality of sentences, and display a described item, which is a property item of a property that is described in at least one of the plurality of sentences among the plurality of property items, on a display screen in an identifiable manner.

In the document creation support apparatus according to the aspect of the present disclosure, the processor may be configured to generate the plurality of sentences in which a combination of the property items of the properties described in the sentences is different.

In the document creation support apparatus according to the aspect of the present disclosure, the processor may be configured to display an undescribed item, which is a property item of a property that is not described in the sentence, on the display screen in an identifiable manner.

In the document creation support apparatus according to the aspect of the present disclosure, the processor may be configured to display the plurality of property items on the display screen and highlight, in response to a selection of any one of the plurality of sentences, the property item corresponding to the described item included in the selected sentence among the plurality of displayed property items.

In the document creation support apparatus according to the aspect of the present disclosure, the processor may be configured to display the plurality of property items on the display screen, and display, in response to a selection of any one of the plurality of sentences, the described item included in the selected sentence and the property item corresponding to the described item included in the selected sentence among the plurality of displayed property items in association with each other.

In the document creation support apparatus according to the aspect of the present disclosure, the processor may be configured to display the plurality of property items in a line in a first region of the display screen and display the plurality of sentences in a line in a second region of the display screen.

In the document creation support apparatus according to the aspect of the present disclosure, the processor may be configured to display the plurality of sentences in a line and display the property item corresponding to the described item in each of the plurality of sentences in close proximity to a corresponding sentence.

“Display in close proximity” means that the sentence and the described item are displayed close to each other so that it can be ascertained that each of the plurality of sentences on the display screen is associated with the described item. Specifically, in a state where a plurality of sentences are displayed in a line, when a distance between a region where a described item of a certain sentence is displayed and a region where a sentence corresponding to the described item is displayed is defined as a first distance, and a distance between the region where the described item is displayed and a region where a sentence not corresponding to the described item is displayed is defined as a second distance, it means that the first distance is smaller than the second distance.

In the document creation support apparatus according to the aspect of the present disclosure, the processor may be configured to display a property item corresponding to an undescribed item in each of the plurality of sentences in a different manner from the property item corresponding to the described item in close proximity to the corresponding sentence.

In the document creation support apparatus according to the aspect of the present disclosure, the processor may be configured to distinguish between an undescribed item, which is a property item of a property that is not described in the selected sentence among the plurality of sentences, and the described item and save the undescribed item and the described item.

In the document creation support apparatus according to the aspect of the present disclosure, the image may be a medical image, and the sentence may be a medical sentence related to the structure of interest included in the medical image.

According to another aspect of the present disclosure, there is provided a document creation support method comprising: deriving properties for each of a plurality of predetermined property items in a structure of interest included in an image; generating a plurality of sentences describing the properties specified for at least one of the plurality of property items; and displaying each of the plurality of sentences, and displaying a described item, which is a property item of the property that is described in at least one of the plurality of sentences among the plurality of property items, on a display screen in an identifiable manner.

In addition, a program for causing a computer to execute the document creation support method according to the aspect of the present disclosure may be provided.

According to the aspects of the present disclosure, it is possible to easily recognize whether or not there is a description of property information about a structure of interest included in an image in a sentence related to the image.

### BRIEF DESCRIPTION OF THE DRAWINGS

FIG. 1 is a diagram showing a schematic configuration of a medical information system to which a document creation support apparatus according to an embodiment of the present disclosure is applied.

FIG. 2 is a diagram showing a schematic configuration of the document creation support apparatus according to the present embodiment.

FIG. 3 is a diagram showing a schematic configuration of the document creation support apparatus according to the present embodiment.

FIG. 4 is a diagram showing an example of supervised training data for training a first learning model.

FIG. 5 is a diagram for describing property information derived by an image analysis unit.

FIG. 6 is a diagram schematically showing a configuration of a recurrent neural network.

FIG. 7 is a diagram showing an example of a display screen of a medical sentence.

FIG. 8 is a diagram showing an example of a display screen of a medical sentence.

FIG. 9 is a diagram showing an example of a display screen of a medical sentence.

FIG. 10 is a diagram for describing saved information.

FIG. 11 is a flowchart showing a process performed in the present embodiment.

FIG. 12 is a diagram showing a display screen in which property items corresponding to undescribed items are displayed.

### DETAILED DESCRIPTION

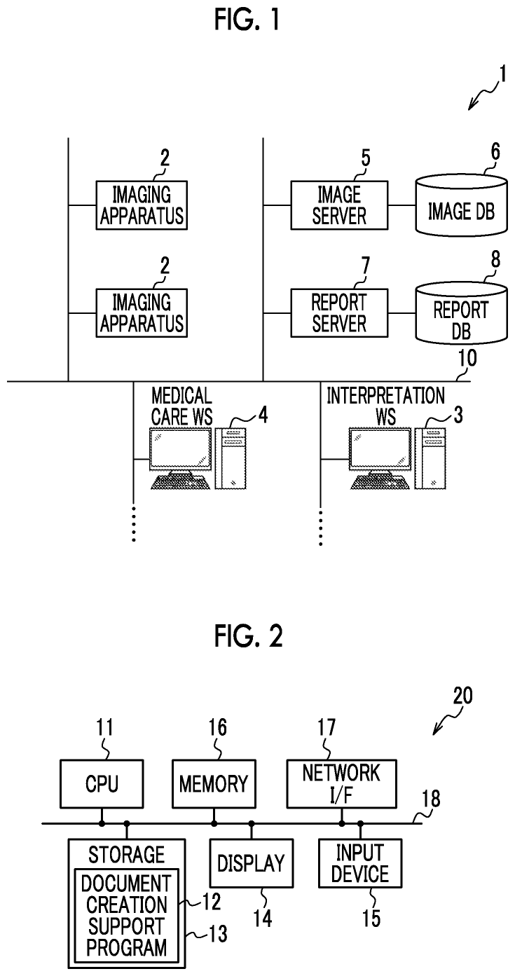

Hereinafter, embodiments of the present disclosure will be described with reference to the drawings. First, a configuration of a medical information system 1 to which a document creation support apparatus according to the present embodiment is applied will be described. FIG. 1 is a diagram showing a schematic configuration of the medical information system 1. The medical information system 1 shown in FIG. 1 is, based on an examination order from a doctor in a medical department using a known ordering system, a system for imaging an examination target part of a subject, storing a medical image acquired by the imaging, interpreting the medical image by a radiologist and creating an interpretation report, and viewing the interpretation report and observing the medical image to be interpreted in detail by the doctor in the medical department that is a request source.

As shown in FIG. 1, in the medical information system 1, a plurality of imaging apparatuses 2, a plurality of interpretation workstations (hereinafter referred to as an interpretation workstation (WS)) 3 that are interpretation terminals, a medical care workstation (hereinafter referred to as a medical care WS) 4, an image server 5, an image database (hereinafter referred to as an image DB) 6, a report server 7, and a report database (hereinafter referred to as a report DB) 8 are communicably connected to each other through a wired or wireless network 10.

Each apparatus is a computer on which an application program for causing each apparatus to function as a component of the medical information system 1 is installed. The application program is stored in a storage apparatus of a server computer connected to the network 10 or in a network storage in a state in which it can be accessed from the outside, and is downloaded to and installed on the computer in response to a request. Alternatively, the optimization support program is recorded on a recording medium, such as a digital versatile disc (DVD) and a compact disc read only memory (CD-ROM), and distributed, and is installed on the computer from the recording medium.

The imaging apparatus 2 is an apparatus (modality) that generates a medical image showing a diagnosis target part of the subject by imaging the diagnosis target part. Specifically, examples of the modality include a simple X-ray imaging apparatus, a CT apparatus, an MRI apparatus, a positron emission tomography (PET) apparatus, and the like. The medical image generated by the imaging apparatus 2 is transmitted to the image server 5 and is saved in the image DB 6.

The interpretation WS 3 is a computer used by, for example, a radiologist of a radiology department to interpret a medical image and to create an image interpretation report, and encompasses a document creation support apparatus 20 according to the present embodiment. In the interpretation WS 3, a viewing request for a medical image to the image server 5, various image processing for the medical image received from the image server 5, display of the medical image, input reception of comments on findings regarding the medical image, and the like are performed. In the interpretation WS 3, an analysis process for medical images and input comments on findings, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by a doctor in a medical department to observe an image in detail, view an interpretation report, create an electronic medical record, and the like, and is configured to include a processing apparatus, a display apparatus such as a display, and an input apparatus such as a keyboard and a mouse. In the medical care WS 4, a viewing request for the image to the image server 5, display of the image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 comprises a storage in which the image DB 6 is configured. This storage may be a hard disk apparatus connected to the image server 5 by a data bus, or may be a disk apparatus connected to a storage area network (SAN) or a network attached storage (NAS) connected to the network 10. In a case where the image server 5 receives a request to register a medical image from the imaging apparatus 2, the image server 5 prepares the medical image in a format for a database and registers the medical image in the image DB 6.

Image data of the medical image acquired by the imaging apparatus 2 and accessory information are registered in the image DB 6. The accessory information includes, for example, an image identification (ID) for identifying each medical image, a patient ID for identifying a subject, an examination ID for identifying an examination, a unique ID (unique identification (UID)) allocated for each medical image, examination date and examination time at which a medical image is generated, the type of imaging apparatus used in an examination for acquiring a medical image, patient information such as the name, age, and gender of a patient, an examination part (an imaging part), imaging information (an imaging protocol, an imaging sequence, an imaging method, imaging conditions, the use of a contrast medium, and the like), and information such as a series number or a collection number in a case where a plurality of medical images are acquired in one examination.

In addition, in a case where the viewing request from the interpretation WS 3 and the medical care WS 4 is received through the network 10, the image server 5 searches for a medical image registered in the image DB 6 and transmits the searched for medical image to the interpretation WS 3 and to the medical care WS 4 that are request sources.

The report server 7 incorporates a software program for providing a function of a database management system to a general-purpose computer. In a case where the report server 7 receives a request to register the interpretation report from the interpretation WS 3, the report server 7 prepares the interpretation report in a format for a database and registers the interpretation report in the report DB 8.

In the report DB 8, an interpretation report including at least the comments on findings created by the radiologist using the interpretation WS 3 is registered. The interpretation report may include, for example, information such as a medical image to be interpreted, an image ID for identifying the medical image, a radiologist ID for identifying the radiologist who performed the interpretation, a lesion name, lesion position information, information for accessing a medical image including a specific region, and property information.

Further, in a case where the report server 7 receives the viewing request for the interpretation report from the interpretation WS 3 and the medical care WS 4 through the network 10, the report server 7 searches for the interpretation report registered in the report DB 8, and transmits the searched for interpretation report to the interpretation WS 3 and to the medical care WS 4 that are request sources.

In the present embodiment, it is assumed that the medical image is a three-dimensional CT image consisting of a plurality of tomographic images with a lung as a diagnosis target, and an interpretation report on an abnormal shadow included in the lung is created as a medical sentence by interpreting the CT image. The medical image is not limited to the CT image, and any medical image such as an MRI image and a simple two-dimensional image acquired by a simple X-ray imaging apparatus can be used.

The network 10 is a wired or wireless local area network that connects various apparatuses in a hospital to each other. In a case where the interpretation WS 3 is installed in another hospital or clinic, the network 10 may be configured to connect local area networks of respective hospitals through the Internet or a dedicated line.

Next, the document creation support apparatus according to the present embodiment will be described. FIG. 2 illustrates a hardware configuration of the document creation support apparatus according to the present embodiment. As shown in FIG. 2, the document creation support apparatus 20 includes a central processing unit (CPU) 11, a non-volatile storage 13, and a memory 16 as a temporary storage area. Further, the document creation support apparatus 20 includes a display 14 such as a liquid crystal display, an input device 15 such as a keyboard and a mouse, and a network interface (I/F) 17 connected to the network 10. The CPU 11, the storage 13, the display 14, the input device 15, the memory 16, and the network I/F 17 are connected to a bus 18. The CPU 11 is an example of a processor in the present disclosure.

The storage 13 is realized by a hard disk drive (HDD), a solid state drive (SSD), a flash memory, and the like. A document creation support program is stored in the storage 13 as a storage medium. The CPU 11 reads a document creation support program 12 from the storage 13, loads the read document creation support program 12 into the memory 16, and executes the loaded document creation support program 12.

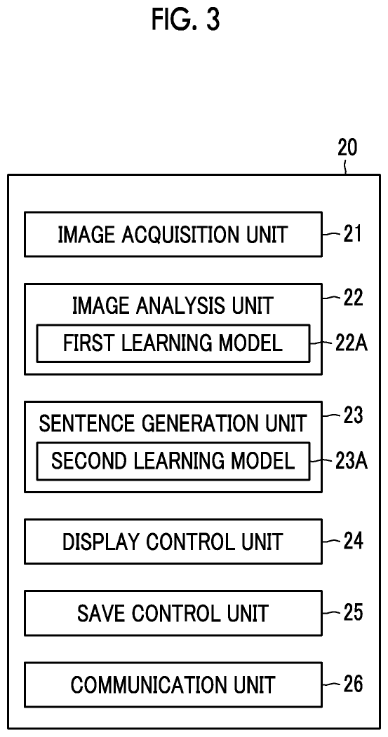

Next, a functional configuration of the document creation support apparatus according to the present embodiment will be described. FIG. 3 is a diagram showing a functional configuration of the document creation support apparatus according to the present embodiment. As shown in FIG. 3, the document creation support apparatus 20 comprises an image acquisition unit 21, an image analysis unit 22, a sentence generation unit 23, a display control unit 24, a save control unit 25, and a communication unit 26. Then, in a case where the CPU 11 executes the document creation support program 12, the CPU 11 functions as the image acquisition unit 21, the image analysis unit 22, the sentence generation unit 23, the display control unit 24, the save control unit 25, and the communication unit 26.

The image acquisition unit 21 acquires a medical image for creating an interpretation report from the image server 5 according to an instruction from the input device 15 by the radiologist who is an operator.

The image analysis unit 22 analyzes the medical image to derive a property for each of a plurality of predetermined property items in the structure of interest included in the medical image. For this purpose, the image analysis unit 22 has a first learning model 22A in which machine learning is performed so as to discriminate an abnormal shadow candidate in the medical image and discriminate the property of the discriminated abnormal shadow candidate. In the present embodiment, the first learning model 22A consists of a convolutional neural network (CNN) in which deep learning is performed using supervised training data so as to discriminate whether or not each pixel (voxel) in the medical image represents an abnormal shadow candidate, and discriminate, in a case where the pixel represents an abnormal shadow candidate, a property for each of a plurality of predetermined property items for the abnormal shadow candidate.

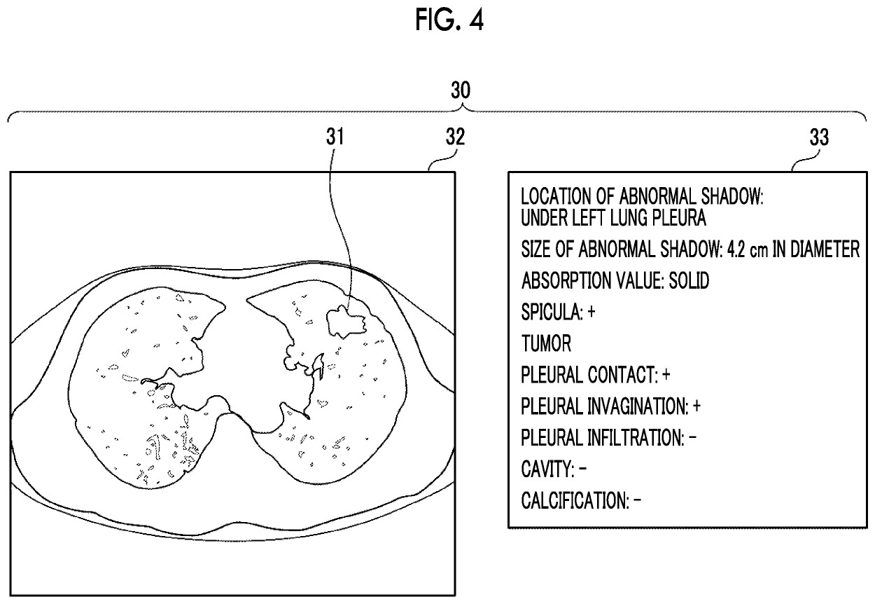

FIG. 4 is a diagram showing an example of supervised training data for training a first learning model. As shown in FIG. 4, supervised training data 30 includes a medical image 32 including an abnormal shadow 31 and property information 33 indicating the property for each of the plurality of property items for the abnormal shadow. In the present embodiment, it is assumed that the abnormal shadow 31 is a lung nodule, and the property information 33 indicates properties for a plurality of property items for the lung nodule. For example, as the property items included in the property information 33, the location of the abnormal shadow, the size of the abnormal shadow, the type of absorption value (solid and frosted glass type), the presence or absence of spicula, whether it is a tumor or a nodule, the presence or absence of pleural contact, the presence or absence of pleural invagination, the presence or absence of pleural infiltration, the presence or absence of a cavity, and the presence or absence of calcification are used. Regarding the abnormal shadow 31 included in the supervised training data 30 shown in FIG. 4, the property information 33 indicates, as shown in FIG. 4, that the location of the abnormal shadow is under the left lung pleura, the size of the abnormal shadow is 4.2 cm in diameter, the absorption value is a solid type, spicula is present, it is a tumor, pleural contact is present, pleural invagination is present, pleural infiltration is absent, a cavity is absent, and calcification is absent. In addition, in FIG. 4, + is given in the case of presence, and − is given in the case of absence. Hereinafter, the case of presence is referred to as a positive finding, and the case of absence is referred to as a negative finding. The first learning model 22A is constructed by training a neural network using a large amount of supervised training data as shown in FIG. 4. For example, by using the supervised training data 30 shown in FIG. 4, the first learning model 22A is trained to discriminate the abnormal shadow 31 included in the medical image 32 in a case where the medical image 32 shown in FIG. 4 is input, and to output the property information 33 shown in FIG. 4 with regard to the abnormal shadow 31.

Further, as the first learning model 22A, any learning model such as, for example, a support vector machine (SVM) can be used in addition to the convolutional neural network.

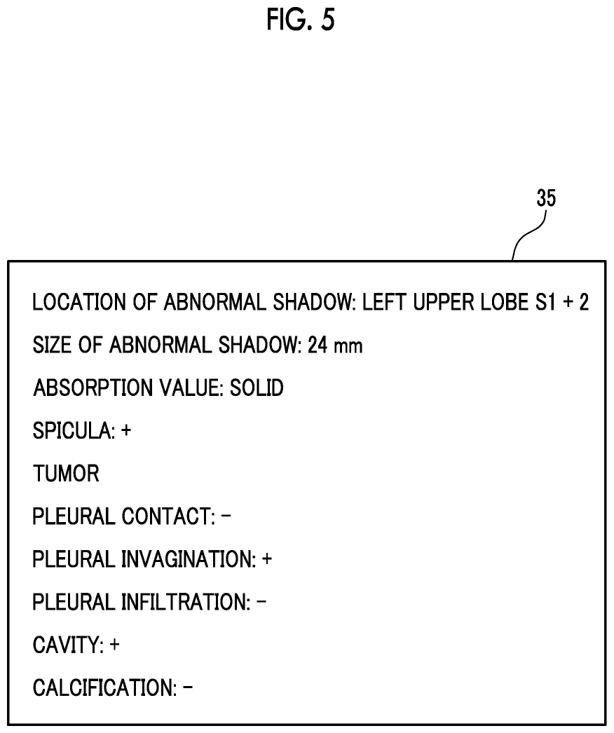

Note that the learning model for detecting the abnormal shadow candidate from the medical image and the learning model for deriving the property information of the abnormal shadow candidate may be constructed separately. Further, the property information derived by the image analysis unit 22 is saved in the storage 13. FIG. 5 is a diagram for describing the property information derived by the image analysis unit 22. As shown in FIG. 5, property information 35 derived by the image analysis unit 22 is assumed to be “left upper lobe S1+S2”, “24 mm”, “solid”, “with spicula”, “tumor”, “no pleural contact”, “with pleural invagination”, “no pleural infiltration”, “with cavity”, and “no calcification” for each of the property items.

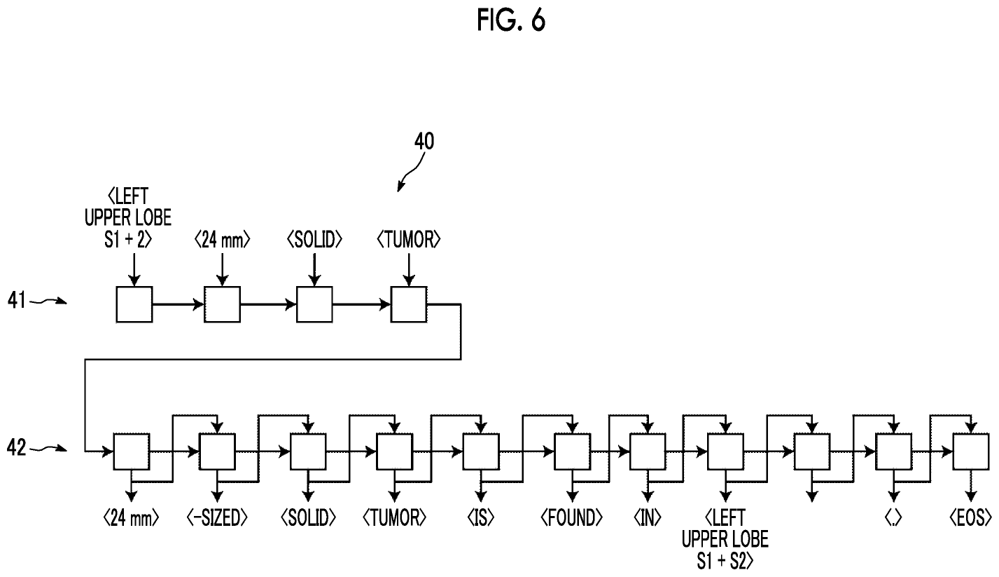

The sentence generation unit 23 generates a medical sentence serving as comments on findings by using the property information derived by the image analysis unit 22. Specifically, the sentence generation unit 23 generates a medical sentence that describes the properties for at least one of the plurality of property items included in the property information derived by the image analysis unit 22. For this purpose, the sentence generation unit 23 consists of a second learning model 23A that has been trained to generate a sentence from the input information. As the second learning model 23A, for example, a recurrent neural network can be used. FIG. 6 is a diagram schematically showing a configuration of a recurrent neural network. As shown in FIG. 6, a recurrent neural network 40 consists of an encoder 41 and a decoder 42. The property information derived by the image analysis unit 22 is input to the encoder 41. For example, property information of “left upper lobe S1+S2”, “24 mm”, “solid”, and “tumor” is input to the encoder 41. The decoder 42 is trained to document character information, and generates a medical sentence from the input property information. Specifically, from the above-mentioned property information of “left upper lobe S1+S2”, “24 mm”, “solid”, and “tumor”, a medical sentence of “A 24 mm-sized solid tumor is found in the left upper lobe S1+S2” is generated. In FIG. 6, “EOS” indicates the end of the sentence (End Of Sentence).

In this way, in order to output the medical sentence by inputting the property information, the recurrent neural network 40 is constructed by learning the encoder 41 and the decoder 42 using a large amount of supervised training data consisting of a combination of the property information and the medical sentence.

Here, in the medical sentence generated by the sentence generation unit 23, at least one of the plurality of property items derived by the image analysis unit 22 is described. The property item described in the sentence generated by the sentence generation unit 23 is referred to as a described item. In addition, a property item that is not described in the medical sentence generated by the sentence generation unit 23 is referred to as an undescribed item.

In the present embodiment, the sentence generation unit 23 generates a plurality of medical sentences describing the properties for at least one of the plurality of property items. For example, in the second learning model 23A, a plurality of medical sentences including a medical sentence generated by inputting all the properties (positive findings and negative findings) specified from the medical image, and a medical sentence generated by inputting only the positive findings, as property items to be input, are generated. Alternatively, a plurality of sentences having a large score indicating the appropriateness of the sentence with respect to the input property information may be generated. In this case, by using index values such as bilingual evaluation understudy (BLEU, see https://qiita.com/inatonix/items/84a66571029334fbc874) as a score indicating the appropriateness of the sentence, a plurality of sentences having a large score can be generated.

For example, as shown in FIG. 5, in a case where the property information 35 derived by the image analysis unit 22 is “left upper lobe S1+S2”, “24 mm”, “solid”, “with spicula”, “tumor”, “no pleural contact”, “with pleural invagination”, “no pleural infiltration”, “with cavity”, and “no calcification” for each of the property items, the sentence generation unit 23 generates, for example, the following three medical sentences.

(1) A 24 mm-sized solid tumor is found in the left upper lobe S1+2. The margin is accompanied by spicula and pleural invagination. A cavity is found inside, but there is no calcification.

(2) A 24 mm-sized solid tumor is found in the left upper lobe S1+2. The margin is accompanied by spicula and pleural invagination. A cavity is found inside.

(3) A 24 mm-sized tumor is found in the left upper lobe S1+2. The margin is accompanied by spicula and pleural invagination. A cavity is found inside.

In the medical sentence (1), the described items are “left upper lobe S1+2”, “24 mm”, “solid”, “tumor”, “spicula: +”, “pleural invagination: +”, “cavity: +”, and “calcification: −”, and the undescribed items are “pleural contact: −” and “pleural infiltration: −”. In the medical sentence (2), the described items are “left upper lobe S1+2”, “24 mm”, “solid”, “tumor”, “spicula: +”, “pleural invagination: +”, and “cavity: +”, and the undescribed items are “pleural contact: −”, “pleural infiltration: −”, and “calcification: −”. In the medical sentence (3), the described items are “left upper lobe S1+2”, “24 mm”, “tumor”, “spicula: +”, “pleural invagination: +”, and “cavity: +”, and the undescribed items are “solid”, “pleural contact: −”, “pleural infiltration: −”, and “calcification: −”.

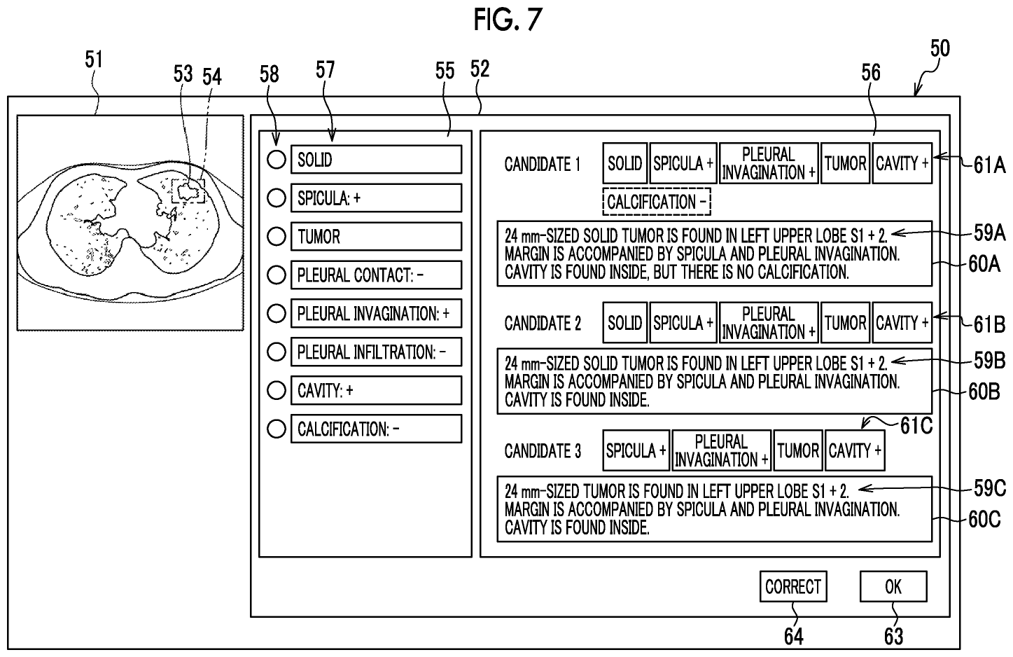

The display control unit 24 displays the medical sentence generated by the sentence generation unit 23 on the display 14. FIG. 7 is a diagram showing an example of a display screen of a medical sentence according to the present embodiment. As shown in FIG. 7, a display screen 50 includes an image display region 51 and an information display region 52. In the image display region 51, a slice image SL1 that is most likely to specify the abnormal shadow candidate detected by the image analysis unit 22 is displayed. The slice image SL1 includes an abnormal shadow candidate 53, and the abnormal shadow candidate 53 is surrounded by a rectangular region 54.

The information display region 52 includes a first region 55 and a second region 56. In the first region 55, a plurality of property items 57 included in the property information derived by the image analysis unit 22 are displayed in a line. On the left side of each property item 57, a mark 58 for indicating the relationship with the described item in the sentence is displayed. The property item 57 includes properties for each property item. In the second region 56, three sentence display regions 60A to 60C for displaying a plurality of (three in the present embodiment) medical sentences 59A to 59C generated by the sentence generation unit 23 in a line are displayed. The titles of candidates 1 to 3 are given to the sentence display regions 60A to 60C, respectively. Further, corresponding property items 61A to 61C corresponding to the described items included in the medical sentences 59A to 59C displayed in each of the sentence display regions 60A to 60C are displayed in close proximity above each of the sentence display regions 60A to 60C, respectively.

A distance between the region where the corresponding property item 61B is displayed and the sentence display region 60B is smaller than a distance between the region where the corresponding property item 61B is displayed and the sentence display region 60A. In addition, a distance between the region where the corresponding property item 61C is displayed and the sentence display region 60C is smaller than a distance between the region where the corresponding property item 61C is displayed and the sentence display region 60B. Therefore, it becomes easy to associate the corresponding property items 61A to 61C with the medical sentences 59A to 59C displayed in the sentence display regions 60A to 60C.

Here, the medical sentence 59A displayed in the sentence display region 60A is the medical sentence (1) described above. The described items of the medical sentence 59A are “left upper lobe S1+2”, “24 mm”, “solid”, “tumor”, “spicula: +”, “pleural invagination: +”, “cavity: +”, and “calcification: −”. Therefore, as the corresponding property item 61A, “solid”, “tumor”, “spicula: +”, “pleural invagination: +”, “cavity: +”, and “calcification: −” other than the location and size of the abnormal shadow are displayed surrounded by solid lines. In the corresponding property item 61A, the frame of “calcification: −”, which is a negative property item, is shown by a broken line so as to clearly indicate that it is negative. In addition, in order to clearly indicate that it is negative, the background color of “calcification: −” may be different from other corresponding property items, or the character size or font may be different from other corresponding property items. The corresponding property item 61A does not include “pleural contact: −” and “pleural infiltration: −” which are the negative property items.

Further, the medical sentence 59B displayed in the sentence display region 60B is the medical sentence (2) described above. The described items of the medical sentence 59B are “left upper lobe S1+2”, “24 mm”, “solid”, “tumor”, “spicula: +”, “pleural invagination: +”, and “cavity: +”. Therefore, as the corresponding property item 61B, “solid”, “tumor”, “spicula: +”, “pleural invagination: +”, and “cavity: +” other than the location and size of the abnormal shadow are displayed surrounded by solid lines. The corresponding property item 61B does not include “pleural contact: −”, “pleural infiltration: −”, and “calcification: −” which are the negative property items.

Further, the medical sentence 59C displayed in the sentence display region 60C is the medical sentence (3) described above. The described items of the medical sentence 59C are “left upper lobe S1+2”, “24 mm”, “tumor”, “spicula: +”, “pleural invagination: +”, and “cavity: +”. Therefore, as the corresponding property item 61C, “tumor”, “spicula: +”, “pleural invagination: +”, and “cavity: +” other than the location and size of the abnormal shadow are displayed surrounded by solid lines. The corresponding property item 61C does not include “pleural contact: −”, “pleural infiltration: −”, and “calcification: −” which are the negative property items. In addition, “solid” property item is not included.

Further, below the second region 56 in the information display region 52, an OK button 63 for confirming the selected medical sentence and a correction button 64 for correcting the selected medical sentence are displayed.

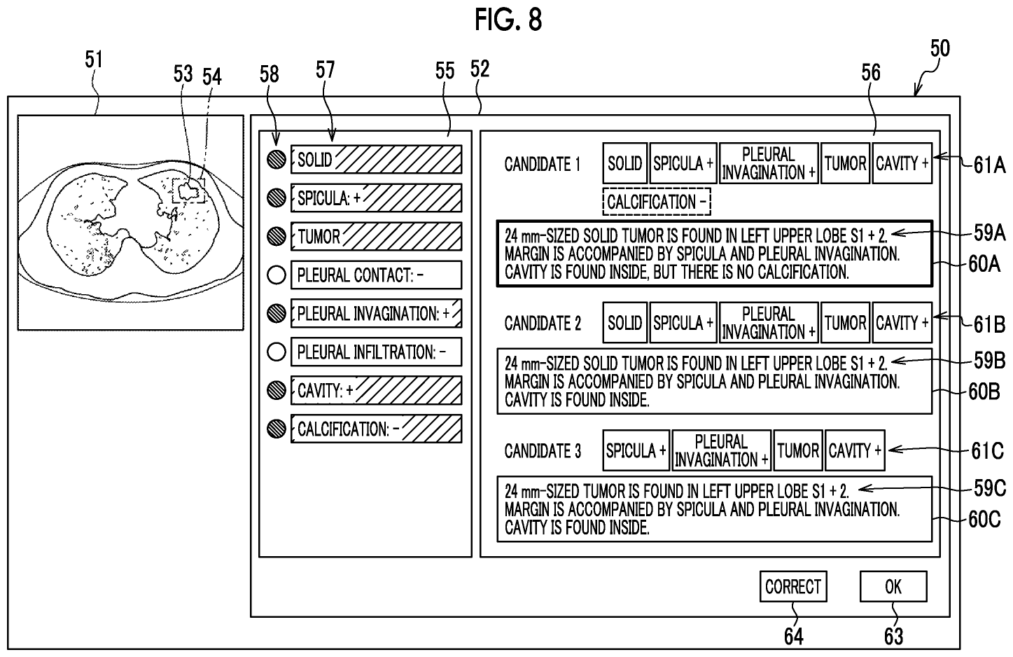

In a case where the radiologist selects any of the sentence display regions 60A to 60B, the property items corresponding to the described items included in the medical sentence displayed in the selected sentence display region among the plurality of property items 57 displayed in the first region 55 are highlighted. For example, as shown in FIG. 8, in a case where the sentence display region 60A is selected, the frame of the sentence display region 60A becomes thicker, and “solid”, “spicula: +”, “tumor”, “pleural invagination: +”, “cavity: +”, and “calcification: −” that are the property items 57 corresponding to the described items of the medical sentence 59A are highlighted. In FIG. 8, the highlighting is shown by giving hatching to each of the property items 57 corresponding to the described items of the medical sentence 59A. For highlighting, it is possible to use a method such as making the color of the property item corresponding to the described item different from other property items, or graying out other property items other than the property item corresponding to the described item. However, the present disclosure is not limited thereto. In addition, in a case where the sentence display region 60A is selected, colors are given to the mark 58 corresponding to each of “solid”, “spicula: +”, “tumor”, “pleural invagination: +”, “cavity: +”, and “calcification: −”. In FIG. 8, the addition of color is shown by filling.

In a case where the sentence display region 60B is selected, “solid”, “spicula: +”, “tumor”, “pleural invagination: +”, and “cavity: +” that are the property items corresponding to the described items of the medical sentence 59B are highlighted in the first region 55. Further, in a case where the sentence display region 60C is selected, “spicula: +”, “tumor”, “pleural invagination: +”, and “cavity: +” that are the property items corresponding to the described items of the medical sentence 59C are highlighted in the first region 55.

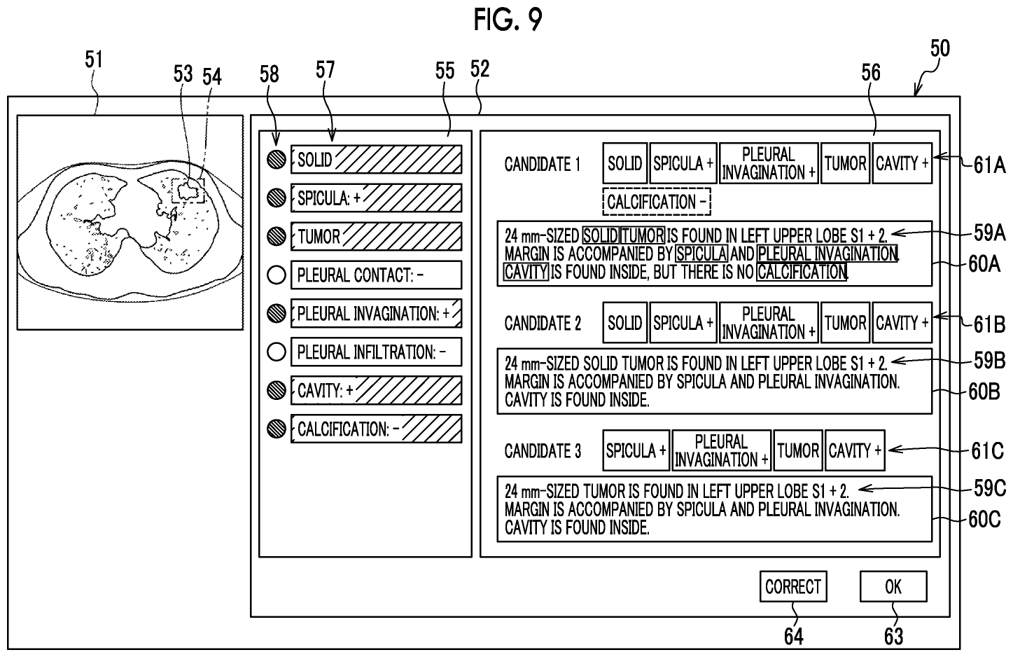

Further, the described item included in the medical sentence displayed in the selected sentence display region and the property items corresponding to the described items included in the medical sentence displayed in the selected sentence display region among the plurality of property items 57 displayed in the first region 55 may be displayed in association with each other. FIG. 9 is a diagram for describing the display of the association between the described item and the property item. As shown in FIG. 9, in a case where the sentence display region 60A is selected, property items of “solid”, “tumor”, “spicula: +”, “pleural invagination: +”, “cavity: +”, and “calcification: −” corresponding to the described items of the medical sentence 59A among the property items 57 displayed in the first region 55 are highlighted. Further, in the medical sentence 59A displayed in the selected sentence display region 60A, the property items of “solid”, “tumor”, “spicula: +”, “pleural invagination: +”, “cavity”, and “calcification: −” described in the medical sentence 59A are highlighted. Accordingly, the described item included in the medical sentence is associated with the property item corresponding to the described item among the plurality of property items 57.

In FIG. 9, the association by highlighting the property item in the medical sentence 59A is represented by enclosing the property item with a solid-line rectangle, but the present disclosure is not limited thereto. For example, by bolding the characters of the property item, changing the color of the characters of the property item, making the character color the same as that of the corresponding property item among the plurality of property items 57 displayed in the first region 55, and the like, the association may be made. Accordingly, the described item included in the sentence displayed in the selected sentence display region and the property items corresponding to the described items included in the sentence displayed in the selected sentence display region among the plurality of property items 57 displayed in the first region 55 are associated with each other.

The radiologist interprets the slice image SL1 displayed in the image display region 51, and determines the suitability of the medical sentences 59A to 59C displayed in the sentence display regions 60A to 60C displayed in the second region 56. In a case where the property item desired by the radiologist is described in the displayed medical sentence, the radiologist selects the sentence display region in which the medical sentence including the desired property item is displayed, and selects the OK button 63. Accordingly, the medical sentence displayed in the selected sentence display region is transcribed in the interpretation report. Then, the interpretation report to which the medical sentence is transcribed is transmitted to the report server 7 together with the slice image SL1 and is stored therein. The interpretation report and the slice image SL1 are transmitted by the communication unit 26 via the network I/F 17.

On the other hand, in a case where the medical sentence displayed in any of the sentence display regions 60A to 60C is not desired by the radiologist, the radiologist selects, for example, one sentence display region and selects the correction button 64. Accordingly, the medical sentence displayed in the selected sentence display regions 60A to 60C can be corrected by using the input device 15. After the correction, in a case where the OK button 63 is selected, the corrected medical sentence is transcribed in the interpretation report. Then, the interpretation report to which the medical sentence is transcribed is transmitted to the report server 7 and is stored therein together with saved information to be described later and the slice image SL1.

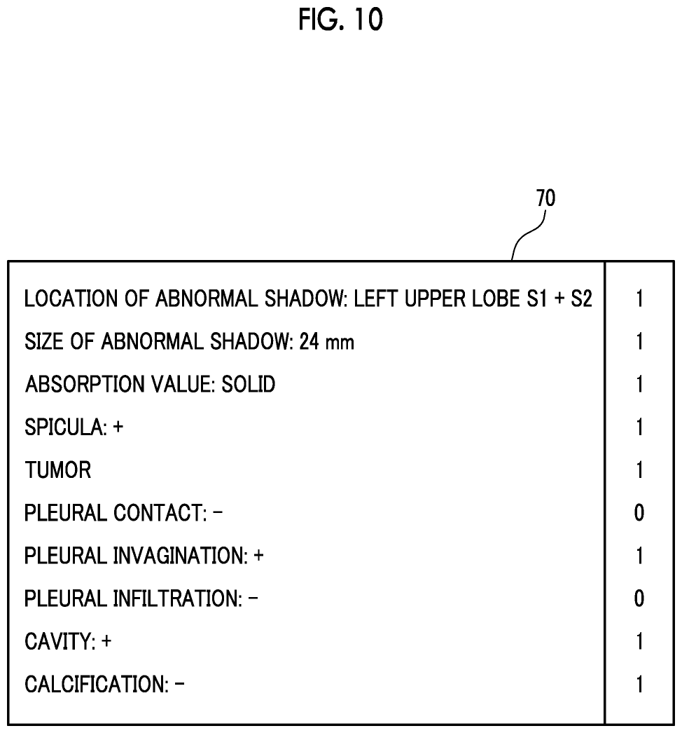

The save control unit 25 distinguishes between undescribed items, which are property items of properties that are not described in the medical sentence displayed in the selected sentence display region, and described items and saves them in the storage 13 as saved information. FIG. 10 is a diagram for describing saved information. For example, in a case where the medical sentence 59A displayed in the sentence display region 60A is selected, the undescribed items are “no pleural contact” and “no pleural infiltration”. As shown in FIG. 10, in saved information 70, a flag of 1 is given to the described item, and a flag of 0 is given to the undescribed item, respectively. The saved information 70 is transmitted to the report server 7 together with the interpretation report as described above.

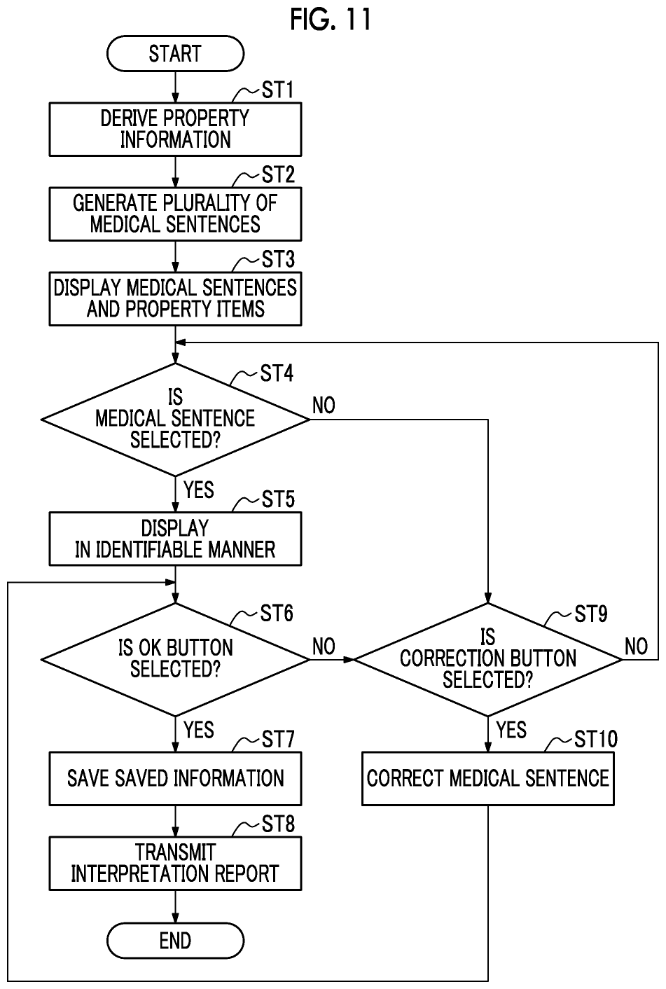

Next, a process performed in the present embodiment will be described. FIG. 11 is a flowchart showing a process performed in the present embodiment. It is assumed that the medical image to be interpreted is acquired from the image server 5 by the image acquisition unit 21 and is saved in the storage 13. The process is started in a case where an instruction to create an interpretation report is given by the radiologist, and the image analysis unit 22 analyzes the medical image to derive property information indicating the property of the structure of interest such as an abnormal shadow candidate included in the medical image (Step ST1). Next, the sentence generation unit 23 generates a plurality of medical sentences related to the medical image based on the property information (Step ST2). Subsequently, the display control unit 24 displays the display screen 50 of a plurality of medical sentences and property items on the display 14 (display of medical sentences and property items: Step ST3).

Then, monitoring of whether or not one medical sentence is selected from the plurality of medical sentences is started (Step ST4). In a case where Step ST4 is affirmative, the described item which is the property item of the property that is described in the selected medical sentence of the plurality of medical sentences among the plurality of property items is displayed in an identifiable manner (display in an identifiable manner: Step ST5).

Subsequently, the display control unit 24 determines whether or not the OK button 63 is selected (Step ST6), and in a case where Step ST6 is affirmative, the save control unit 25 distinguishes between undescribed items, which are property items of properties that are not described in the selected medical sentence, and described items and saves them in the storage 13 as the saved information 70 (saving saved information: Step ST7). Further, the display control unit 24 transcribes the selected sentence to the interpretation report, the communication unit 26 transmits the interpretation report to which the sentence is transcribed to the report server 7 together with the slice image SL1 (transmission of interpretation report: Step ST8), and the process ends.

In a case where Step ST4 and Step ST6 are negative, the display control unit 24 determines whether or not the correction button 64 is selected (Step ST9). In a case where Step ST9 is negative, the process returns to Step ST4, and the processes after Step ST4 are repeated. In a case where Step ST9 is affirmative, the display control unit 24 receives the correction of the selected medical sentence, the selected medical sentence is corrected accordingly (Step ST10), the process proceeds to Step ST6, and the processes after Step ST6 are repeated.

As described above, in the present embodiment, it is configured to display each of the plurality of medical sentences, and display a described item, which is a property item of the property that is described in at least one of the plurality of medical sentences among the plurality of property items, on the display screen 50 in an identifiable manner. Therefore, it is possible to easily recognize whether or not there is a description of property information about a structure of interest included in a medical image in a medical sentence.

Further, by displaying an undescribed item, which is a property item of the property that is not described in the medical sentence, in an identifiable manner, the property item that is not described in the displayed medical sentence can be easily recognized.

In addition, by displaying a plurality of property items and highlighting, in response to a selection of any one of the plurality of medical sentences, the property item corresponding to the described item included in the selected medical sentence among the plurality of displayed property items, it is possible to easily recognize which property item is described in the selected medical sentence.

In addition, by displaying a plurality of property items and displaying, in response to a selection of any one of the plurality of medical sentences, the described item included in the selected medical sentence and the property item corresponding to the described item included in the selected medical sentence among the plurality of displayed property items in association with each other, it is possible to easily recognize which of the plurality of displayed property items the property item described in the medical sentence is associated with.

In addition, by displaying a plurality of medical sentences in a line and displaying the property items corresponding to the described items in each of the plurality of medical sentences in close proximity to the corresponding medical sentences, it becomes easy to associate the displayed medical sentence with the property item corresponding to the described item in the medical sentence.

Further, by distinguishing between the undescribed items, which are the property items of the property that is not described in the medical sentence displayed in the selected sentence display region, and the described items and saving them as the saved information 70, for example, the saved information 70 can be used as supervised training data at the time of learning the recurrent neural network applied to the sentence generation unit 23. That is, by using the sentence in a case where the saved information 70 is generated and the saved information as supervised training data, it is possible to learn the recurrent neural network so as to give priority to the described items and generate the medical sentence. Therefore, it is possible to learn a recurrent neural network so that a medical sentence that reflects the preference of a radiologist can be generated.

In the above embodiment, the corresponding property items 61A to 61C corresponding to the described items included in the medical sentences 59A to 59C displayed in each of the sentence display regions 60A to 60C are displayed in close proximity to each piece of information in the sentence display regions 60A to 60C. However, the present disclosure is not limited thereto. The property items corresponding to the undescribed items that are not included in the medical sentences 59A to 59C respectively displayed in the sentence display regions 60A to 60C may be displayed as non-corresponding property items in a different manner from the corresponding property items 61A to 61C in close proximity to each of the sentence display regions 60A to 60C.

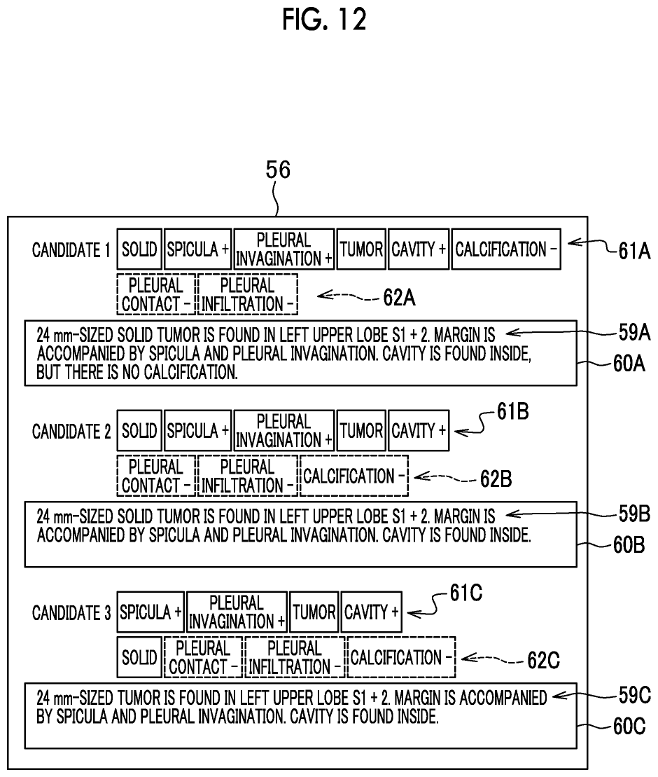

FIG. 12 is a diagram showing a display screen in which property items corresponding to undescribed items are displayed. Further, in FIG. 12, only the second region 56 shown in FIG. 7 is shown. As shown in FIG. 12, the plurality of sentence display regions 60A to 60C on which each of the medical sentences 59A to 59C is displayed are displayed in the second region 56, and the corresponding property items 61A to 61C and the non-corresponding property items 62A to 62C are displayed in the vicinity of each of the sentence display regions 60A to 60C. The corresponding property items 61A to 61C are surrounded by solid-line rectangles, and the non-corresponding property items 62A to 62C are surrounded by broken-line rectangles. Accordingly, the non-corresponding property items 62A to 62C are displayed in a different manner from the corresponding property items 61A to 61C. The mode of display of the corresponding property items 61A to 61C and the non-corresponding property items 62A to 62C is not limited thereto. In the corresponding property items 61A to 61C and the non-corresponding property items 62A to 62C, only the non-corresponding property items 62A to 62C may be grayed out, or the background color may be changed between the corresponding property items 61A to 61C and the non-corresponding property items 62A to 62C.

In this way, by displaying the non-corresponding property items 62A to 62C in a different manner from the corresponding property items 61A to 61C, it becomes easy to associate the displayed medical sentence with the property item corresponding to the described item and to the undescribed item in the medical sentence.

In the above embodiment, a plurality of medical sentences are generated from the medical image, but only one sentence may be generated. In this case, only one sentence display region is displayed in the second region 56 of the display screen 50.

Further, in the above embodiment, the creation support process for the medical sentence such as the interpretation report is performed by generating the medical sentence using the medical image with the lung as the diagnosis target, but the diagnosis target is not limited to the lung. In addition to the lung, any part of a human body such as a heart, liver, brain, and limbs can be diagnosed. In this case, for each learning model of the image analysis unit 22 and of the sentence generation unit 23, learning models that perform the analysis process and the sentence generation process according to the diagnosis target are prepared, a learning model that performs the analysis process and the sentence generation process according to the diagnosis target is selected, and a process of generating a medical sentence is executed.

In addition, in the above embodiment, although the technology of the present disclosure is applied to the case of creating an interpretation report as a medical sentence, the technology of the present disclosure can also be applied to a case of creating medical sentences other than the interpretation report, such as an electronic medical record and a diagnosis report.

Further, in the above embodiment, the medical sentence is generated using the medical image, but the present disclosure is not limited thereto. Of course, the technology of the present disclosure can also be applied even in a case where a sentence relating to any image other than a medical image is generated.

Further, in the above embodiment, for example, as hardware structures of processing units that execute various kinds of processing, such as the image acquisition unit 21, the image analysis unit 22, the sentence generation unit 23, the display control unit 24, the save control unit 25, and the communication unit 26, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example where a plurality of processing units are configured by one processor, first, there is a form in which one processor is configured by a combination of one or more CPUs and software as typified by a computer, such as a client or a server, and this processor functions as a plurality of processing units. Second, there is a form in which a processor for realizing the function of the entire system including a plurality of processing units via one integrated circuit (IC) chip as typified by a system on chip (SoC) or the like is used. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

What is claimed is:

1. A document creation support apparatus comprising at least one processor,
wherein the processor is configured to
derive properties for each of a plurality of predetermined property items in a structure of interest included in an image,
generate a plurality of sentences describing the properties specified for at least one of the plurality of property items, and
display each of the plurality of sentences, and display a described item, which is a property item of a property that is described in at least one of the plurality of sentences among the plurality of property items, on a display screen in an identifiable manner.

2. The document creation support apparatus according to claim 1, wherein the processor is configured to generate the plurality of sentences in which a combination of the property items of the properties described in the sentences is different.

3. The document creation support apparatus according to claim 1, wherein the processor is configured to display an undescribed item, which is a property item of a property that is not described in the sentence, on the display screen in an identifiable manner.

4. The document creation support apparatus according to claim 1, wherein the processor is configured to display the plurality of property items on the display screen and highlight, in response to a selection of any one of the plurality of sentences, the property item corresponding to the described item included in the selected sentence among the plurality of displayed property items.

5. The document creation support apparatus according to claim 1, wherein the processor is configured to display the plurality of property items on the display screen, and display, in response to a selection of any one of the plurality of sentences, the described item included in the selected sentence and the property item corresponding to the described item included in the selected sentence among the plurality of displayed property items in association with each other.

6. The document creation support apparatus according to claim 4, wherein the processor is configured to display the plurality of property items in a line in a first region of the display screen and display the plurality of sentences in a line in a second region of the display screen.

7. The document creation support apparatus according to claim 1, wherein the processor is configured to display the plurality of sentences in a line and display the property item corresponding to the described item in each of the plurality of sentences in close proximity to a corresponding sentence.

8. The document creation support apparatus according to claim 7, wherein the processor is configured to display a property item corresponding to an undescribed item in each of the plurality of sentences in a different manner from the property item corresponding to the described item in close proximity to the corresponding sentence.

9. The document creation support apparatus according to claim 1, wherein the processor is configured to distinguish between an undescribed item, which is a property item of a property that is not described in the selected sentence among the plurality of sentences, and the described item and save the undescribed item and the described item.

10. The document creation support apparatus according to claim 1, wherein the image is a medical image, and the sentence is a medical sentence related to the structure of interest included in the medical image.

11. A document creation support method comprising:
deriving properties for each of a plurality of predetermined property items in a structure of interest included in an image;
generating a plurality of sentences describing the properties specified for at least one of the plurality of property items; and
displaying each of the plurality of sentences, and displaying a described item, which is a property item of a property that is described in at least one of the plurality of sentences among the plurality of property items, on a display screen in an identifiable manner.

12. A non-transitory computer-readable storage medium that stores a document creation support program for causing a computer to execute a procedure comprising:
deriving properties for each of a plurality of predetermined property items in a structure of interest included in an image;
generating a plurality of sentences describing the properties specified for at least one of the plurality of property items; and
displaying each of the plurality of sentences, and displaying a described item, which is a property item of a property that is described in at least one of the plurality of sentences among the plurality of property items, on a display screen in an identifiable manner.
